SEQUENCE LISTING (10 sequences)

SEQ ID NO: 1 - DNA, 21 bp, Artificial Sequence
  Description of Artificial Sequence Synthetic oligonucleotide
  modified_base (10)..(10): Abasic site
atcgtcgagnagttacaagct

SEQ ID NO: 2 - DNA, 16 bp, Artificial Sequence
  Description of Artificial Sequence Synthetic oligonucleotide
ttgcgatgtctaatgg

SEQ ID NO: 3 - DNA, 16 bp, Artificial Sequence
  Description of Artificial Sequence Synthetic oligonucleotide
ttgcgaagtctaatgg

SEQ ID NO: 4 - DNA, 16 bp, Artificial Sequence
  Description of Artificial Sequence Synthetic oligonucleotide
  modified_base (7)..(7): a, c, t, or g
ttgcgangtctaatgg

SEQ ID NO: 5 - DNA, 18 bp, Artificial Sequence
  Description of Artificial Sequence Synthetic oligonucleotide
  modified_base (10)..(10): Abasic site
atcgtcgagnagtacagc

SEQ ID NO: 6 - DNA, 20 bp, Artificial Sequence
  Description of Artificial Sequence Synthetic oligonucleotide
  modified_base (10)..(10): Abasic site
atcgtcgagnagttacaagc

SEQ ID NO: 7 - DNA, 240 bp, Artificial Sequence
  Description of Artificial Sequence Synthetic polynucleotide
tttactctcacatcctgtagtgattgacactgcaacagccaccatcactagaagaacagaacaattacttaatagaaaaattatatcttcctcgaaacgatttcctgcttccaacatctacgtatatcaagaagcattcacttaccatgacacagcttcagatttcattattgctgacagctactatatcactactccatctagtagtggccacgccctatgaggcatatcctatcggaa

SEQ ID NO: 8 - DNA, 300 bp, Artificial Sequence
  Description of Artificial Sequence Synthetic polynucleotide
tatctgatgcgaacaccacgttgtatttcaatgtaatactcgagggtacggactctgccgacagcacgtctttgaacaatacataccaatttgttgttacaaaccgtccatccatctcgctatcgtcagatttcaatctattggcgttgttaaaaaactatggttatactaacggcaaaaacgctctgaaactagatcctaatgaagtcttcaacgtgacttttgaccgttcaatgttcactaacgaagaatccattgtgtcgtattacggacgttctcagttgtataatgcgccgttac

SEQ ID NO: 9 - DNA, 300 bp, Artificial Sequence
  Description of Artificial Sequence Synthetic polynucleotide
ctaacgaagaatccattgtgtcgtattacggacgttctcagttgtataatgcgccgttacccaattggctgttcttcgattctggcgagttgaagtttactgggacggcaccggtgataaactcggcgattgctccagaaacaagctacagttttgtcatcatcgctacagacattgaaggattttctgccgttgaggtagaattcgaattagtcatcggggctcaccagttaactacctctattcaaaatagtttgataatcaacgttactgacacaggtaacgtttcatatgacttac

SEQ ID NO: 10 - DNA, 300 bp, Artificial Sequence
  Description of Artificial Sequence Synthetic polynucleotide
atttgctcagagttcaaatcggcctctttcagtttatccattgcttccttcagtttggcttcactgtcttctagctgttgttctagatcctggtttttcttggtgtagttctcattattagatctcaagttattggagtcttcagccaattgctttgtatcagacaattgactctctaacttctccacttcactgtcgagttgctcgtttttagcggacaaagatttaatctcgttttctttttcagtgttagattgctctaattctttgagctgttctctcagctcctcatatttttct

(19) United States
(12) Patent Application Publication
(10) Pub. No.: US 2019/0304571 A1
(43) Pub. Date: Oct. 3, 2019

(54) SYSTEMS AND METHODS FOR BIOLOGICAL DATA MANAGEMENT

(71) Applicant: Quantum Biosystems Inc., Tokyo (JP)

(72) Inventors: Masoud Vakili, Los Altos, CA (US); Kurt Christofferson, Santa Rosa, CA (US); Mark Oldham, Emerald Hills, CA (US)

(21) Appl. No.: 16/156,755

(22) Filed: Oct. 10, 2018

Related U.S. Application Data
(63) Continuation of application No. PCT/JP2017/014847, filed on Apr. 11, 2017.
(60) Provisional application No. 62/321,103, filed on Apr. 11, 2016.

(51) Int. Cl. G16B 50/30; G16B 50/40; G06F 17/18

(52) U.S. Cl. CPC G16B 50/30; G06F 17/18; G16B 50/40

(57) ABSTRACT

Systems and methods for biological data management may preserve alternative interpretations of data and may implement multi-level encryption and privacy management. Systems and methods for biological data management may include a cell-level architecture, a bank-and-bloc-level architecture, and/or a multi-tiered architecture. Systems and methods for biological data management may incorporate definitions, rules, and directives and/or employ a two-dimensional or three-dimensional data structure.

### CROSS-REFERENCE

This application claims priority to U.S. Provisional Patent Application No. 62/321,103, filed Apr. 11, 2016, which is entirely incorporated herein by reference.

### BACKGROUND ART

New research continues to increase our understanding of genetic information and raise challenges about how to manage such information. A more complete understanding of genetic maps with a higher level of resolution may render valuable results in healthcare and other disciplines.

As an example, one of the challenges in managing genetic deoxyribonucleic acid (DNA) data is that there are highly conserved regions of code, which remain unchanged over time, yet do not seem to code proteins. Research indicates, however, that they may play important roles in gene expression regulation, alternative splicing, and distal enhancers. An efficient way to save regions that are utilized infrequently, while sustaining fast access to more frequently used regions of a genetic sequence, is therefore desirable.

### SUMMARY OF INVENTION

Recognized herein is a need for data management schemes that can accommodate alternative interpretations of data and hence may have access to lower-level data measured by various devices. Also recognized herein is a need to sense, store, and manage genetic data with greater flexibility and greater completeness, as well as a need to flexibly and efficiently create, add to, maintain, and query these data sets at different levels while handling error scenarios.

Provided herein are systems and methods for efficiently and securely managing genetic data, including reading and interpreting raw data, storing and interpreting the genetic data, and maintaining privacy and confidentiality of the data.

Some systems and methods may provide definitions and rules, and issue appropriate directives for issues related to healthcare, food safety, and/or other pathogen handling situations. A multi-tier network architecture in an information handling environment may be utilized.

Parallelism may be used as required by the task and type of biological data interpretation. Information may be initially stored in a distributed storage of semi-structured data, allowing for scanning, reducing, and reorganizing information as needed into structured, columnar, or relational databases.

Systems and methods may stage and perform different queries concurrently, allowing information to be stored in repositories, and may be encrypted at rest. Information may be transmitted across a distributed system, between repositories, between servers, or between servers and clients in an secure and flexible fashion.

Systems and methods can store biological data in one or more storage devices according to a relationship between a size of data or units of data and a size of unit storage blocks or banks of one or more storage devices.

Systems and methods may support access controls, which may be user, role, application, process, or location based.

Systems and methods may relate to mapping and storing genetic data, (e.g., polynucleotide data) in one or more memory devices at a memory cell level, at a memory block level, at a memory bank level, or at another memory partition level.

An aspect of the present disclosure provides a biological data management system, comprising: (a) an end-user module comprising a sequencing device, the sequencing device configured to generate base data; (b) a local repository in network communication with the end-user module, the local repository programmed or configured to (i) receive the base data, (ii) convert the base data into sequence data, (iii) produce abbreviated data based on the sequence data, and (iv) compare the abbreviated data with a database of existing abbreviations; and (c) a central server in network communication with the local repository, the central server configured to update the database of the existing abbreviations.

In some embodiments, the local repository is further programmed or configured to flag abbreviations and communicate the flagged abbreviations to the central server. In some embodiments, the central server is further programmed or configured to receive a flagged abbreviation and perform further analysis on the flagged abbreviation. In some embodiments, the central server is further programmed or configured to generate a directive and communicate the directive to the local repository upon the analysis of the flagged abbreviation. In some embodiments, the abbreviation is a variance, hash, or a checksum.

Another aspect of the present disclosure provides a method for storing biological data, comprising: (a) determining a size of the biological data to identify a storage unit size suitable to store the biological data; (b) identifying a memory location in a memory device having a block size compatible with the storage unit size; and (c) storing the biological data in an erasable block at the memory location of the memory device.

In some embodiments, each erasable block comprises a section for storing the biological data and a section for storing metadata related to the biological data. In some embodiments, the section for storing metadata comprises a longer lifetime. In some embodiments, the section for storing metadata comprises a controller different from a controller of the section for storing sequence data. In some embodiments, the section for storing metadata is configured for more frequent access than the section for storing sequence data.

Another aspect of the present disclosure provides a biological data management system, comprising: (a) a first memory device configured to store biological data for infrequent access; and (b) a second memory device having a block size, the second memory device being in communication with the first memory device and configured to store biological data for frequent access; wherein the second memory device is faster than the first memory device, and wherein the block size is selected to store the biological data according to a size of the biological data.

In some embodiments, the biological data is an n-mer sequence, and the block size is n times a number of bits required to store a monomer of the n-mer. In some embodiments, the biological data is an n-mer sequence, and the block size is at least n times a number of bits required to store a monomer of the n-mer. In some embodiments, the second memory device comprises a flash memory device. In some embodiments, the second memory device comprises a block that is a flash memory erase block.

Another aspect of the present disclosure provides a method for storing sequence base data in a multi-level cell (MLC) memory device, the MLC memory device comprising memory cells, each of the memory cells configured to store two bits, the method comprising, in a memory cell: (a) setting the two bits to 00 to represent a base of a first type; (b) setting the two bits to 01 to represent a base of a second type; (c) setting the two bits to 10 to represent a base of a third type; or (d) setting the two bits to 11 to represent a base of a fourth type.

In some embodiments, the sequence base data represents one or more polynucleotides, each of the polynucleotides comprising one or more bases, each of the one or more bases being one of at least four possible bases. In some embodiments, the polynucleotide is a DNA or an RNA.

Another aspect of the present disclosure provides a method for storing biological data in a memory device, the memory device comprising blocks, each of the blocks comprising a block size, the method comprising: (a) determining a size of the biological data; (b) determining a block size of at least a subset of the blocks; (c) compressing the biological data based on the block size to produce compressed biological data; and (d) storing the biological data in the at least a subset of the blocks.

The method of claim 19, wherein the memory device comprises a flash memory device, and wherein the block size is an erase block size.

In some embodiments, the block size is greater than or equal to a size of the compressed biological data. In some embodiments, the erase block stores the biological data and metadata of the biological data.

Another aspect of the present disclosure provides a method for storing sequence base data in a memory device, the memory device comprising memory cells, each of the memory cells configured to store at least three bits, the method comprising, in a memory cell: (a) setting three of the at least three hits to 000 to represent a base of a first type; (b) setting three of the at least three bits to 001 to represent a base of a second type; (c) setting three of the at least three bits to 010 to represent a base of a third type; (d) setting three of the at least three bits to 011 to represent a base of a fourth type; (e) setting three of the at least three bits to 100 to represent a base of a fifth type; (f) setting three of the at least three bits to 101 to represent a base of a sixth type; (g) setting three of the at least three bits to 110 to represent a base of a seventh type; and (h) setting three of the at least three bits to 111 to represent a base of an eighth type.

In some embodiments, the sequence base data represents one or more polynucleotides, each of the polynucleotides comprising one or more bases, each of the one or more bases being one of four different native bases, a methylated base, an oxidated base, or an abasic location. In some embodiments, the polynucleotide is a DNA or an RNA. In some embodiments, the memory device comprises a flash memory, a phase-change memory, or a resistive memory.

Another aspect of the present disclosure provides a method for storing sequence base data in a memory device, the sequence base data comprising two probable bases to represent each of a plurality of bases measured, the memory device comprising memory cells, each of the memory cells configured to store a plurality of bits, the method comprising: storing in a first bit of the plurality of bits a most probable base of the sequence base data; storing in a second bit of the plurality of bits a second most probable base of the sequence base data; and storing in a remainder of the plurality of bits a relative probability of the most probable base and the second most probable base.

In some embodiments, the method further comprises, using a first cell of the memory cells to identify the most probable base; using a second cell of the memory cells to identify the second most probable base; and using one or more other cells of the memory cells to store the relative probability. In some embodiments, the method further comprises storing in a third cell of the memory cells a probability of the second most probable base.

Another aspect of the present disclosure provides a method for storing sequence base data in a memory device comprising memory cells each configured to store at least three bits, the method comprising, in a memory cell: (a) providing a first bit indication comprising three bits of the at least three bits to represent a base of a first type; (b) providing a second bit indication comprising three bits of the at least three bits to represent a base of a second type; (c) providing a third bit indication comprising three bits of the at least three bits to represent a base of a third type; (d) providing a fourth bit indication comprising three bits of the at least three bits to represent a base of a fourth type; (e) providing a fifth bit indication comprising three bits of the at least three hits to represent a methylated base; (f) providing a sixth bit indication comprising three bits of the at least three bits to represent an oxidated base; and (g) providing a seventh bit indication comprising three bits of the at least three bits to represent an abasic site.

In some embodiments, the memory device comprises a flash memory, a phase-change memory, or a resistive memory.

Another aspect of the present disclosure provides a method for encrypting biological sequence data, the method comprising: (a) identifying a normal level of variance in the biological sequence data; and (b) introducing a second level of variation into the biological sequence data, the second level of variation comparable to the normal level of variance, such that the biological sequence data is indistinguishable with respect to the normal level of variance.

In some embodiments, the method further comprises communicating the introduced level of variance using an encryption method.

Another aspect of the present disclosure provides a method for encrypting biological sequence data of a subject, the method comprising: (a) encrypting information related to the subject using a first encryption scheme; and (b) encrypting the biological sequence data using a second encryption scheme, which second encryption scheme is different from the first encryption scheme.

In some embodiments, the second encryption scheme comprises a less extensive encryption than the first encryption scheme. In some embodiments, the second encryption scheme comprises chaffing and winnowing. In some embodiments, the first encryption scheme uses a public key infrastructure and the second encryption scheme uses the public key infrastructure. In some embodiments, the first encryption scheme uses a first public key infrastructure and the second encryption scheme uses a second public key infrastructure different from the first public key infrastructure.

Another aspect of the present disclosure provides a method for storing sequence base data, the method comprising: providing a two-dimensional table structure in computer memory, the two-dimensional table structure configured to store information representing potential bases; storing information representing the most probable measured bases of the sequence base data in a first dimension of the two-dimensional table structure; storing information representing other potential bases of the sequence base data in a second dimension of the two-dimensional table structure; and storing probabilities corresponding to an intersection of the first dimension and the second dimension in the two-dimensional table structure.

In some embodiments, the potential bases comprise a set of each of four possible bases and at least one of a methylated base, an oxidated base, and an abasic site. In some embodiments, the method further comprises providing a second two-dimensional table structure in computer memory, the second two-dimensional table structure configured to store information representing potential bases; and storing in the second two-dimensional table structure the most probable measured bases of the sequence base data and the second most probable measured bases of the sequence base data.

Another aspect of the present disclosure provides a method for managing biological data, the method comprising: providing an application server programmed or configured to (i) receive raw measured biological data from a sensor and (ii) generate processed biological data from the raw measured biological data; receiving, at the application server, from a local repository, definitions and rules related to the processed biological data; and issuing, by the application server, directives based on the definitions and rules related to the processed biological data.

In some embodiments, the processed biological data comprises a portion of the processed biological data for which related definitions and rules are not found in the local repository, and the method further comprises sending at least the portion of the processed biological data to the local repository. In some embodiments, the method further comprises sending at least the portion of the processed biological data from the local repository to a central server. In some embodiments, the method further comprises sending directives from the central server to the local repository. In some embodiments, the method further comprises sending new definitions and rules from the central server to the local repository.

Another aspect of the present disclosure provides a method for storing sequence base data, the method comprising: for a base location, storing information representing a most probable base of the sequence base data in a first location of a storage device, and storing a probability of a number of occurrences of the most probable base in a second location of the storage device.

Another aspect of the present disclosure provides a method for storing sequence base data comprising at least four possible bases, the method comprising: (a) providing a three-dimensional table structure in computer memory, which three-dimensional table structure is configured to store the sequence base data, wherein (i) a first dimension of the three-dimensional table structure stores information representing most probable measured bases of the genetic sequence base data; (ii) a second dimension of the three-dimensional table structure stores information representing potential bases of the genetic sequence base data; and (iii) a third dimension of the three-dimensional table structure stores information representing a base count probability for each of the at least four possible bases of the sequence base data; (b) storing probabilities corresponding to an intersection of the first dimension, the second dimension, and the third dimension in the three-dimensional table structure.

Another aspect of the present disclosure provides a method for protecting biological data related to a subject, the method comprising: encrypting personal identification information of the subject using a first encryption scheme; encrypting phenotypes of the subject using a second encryption scheme; encrypting the biological data using a third encryption scheme, wherein the second encryption scheme or the third encryption scheme is different from the first encryption scheme; and storing the encrypted personal identification information, the encrypted phenotypes, and the encrypted biological data in computer memory.

In some embodiments, (i) the second encryption scheme is different from the first encryption scheme, and (ii) the third encryption scheme is different from the first encryption scheme, and (iii) the third encryption scheme is different from the second encryption scheme. In some embodiments, the method further comprises storing gene expression data of the subject. In some embodiments, the method further comprises storing geographic data of the subject.

Another aspect of the present disclosure provides a method for storing genetic data of a subject, the method comprising: storing personal identification information of the subject in a first storage segment with a first level of limitation of access; storing phenotype data of the subject in a second storage segment with a second level of limitation of access; and storing the genetic data of the subject in a third storage segment with a third level of limitation of access.

In some embodiments, the second level of limitation of access or the third level of limitation of access is different from the first level of limitation of access. In some embodiments, (i) the second level of limitation of access is different from the first level of limitation of access, and (ii) the third level of limitation of access is different from the first level of limitation of access, and (iii) the third level of limitation of access is different from the second level of limitation of access.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings (also “figure” and “MG.” herein), of which:

FIG. 1 illustrates an example of a conductance-time profile of a sensor.

FIG. 2 illustrates an example of a schematic of a biological data management system.

FIG. 3 illustrates an example of a diagram of a distributed network for biological data management.

FIG. 4 illustrates an example of a schematic of a biological data management system where the central server is sitting in a central location.

FIG. 5 illustrates an example of a flow chart illustrating processes that can be executed by an application server.

FIG. 6 illustrates an example of a flow chart illustrating processes that can be executed by a local repository.

FIG. 7 illustrates an example of a base probability matrix for a 21-mer reading by a sensor.

FIG. 8 illustrates an example of additional dimensions of data kept for a read.

FIG. 9 illustrates examples of various sample identifiers.

FIG. 10 illustrates three examples of syntaxes.

FIG. 11 illustrates an example of a transitional syntax.

FIG. 12 illustrates an example of an application server input.

FIG. 13 illustrates an example of an application server output.

FIG. 14 illustrates an example of a distributed file system.

FIG. 15 illustrates an example of an architecture for segmented access control.

FIGS. 16A, 16B, 16C, and 16D illustrate examples of a tiered storage access schemes.

FIGS. 16A, 16B, 16C, and 16D illustrate examples of a tiered storage access schemes.

FIGS. 16A, 16B, 16C, and 16D illustrate examples of a tiered storage access schemes.

FIGS. 16A, 16B, 16C, and 16D illustrate examples of a tiered storage access schemes.

FIG. 17 illustrates an example of a computer system programmed or otherwise configured to manage biological data.

### DESCRIPTION OF EMBODIMENTS

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

The term “subject,” as used herein, generally refers to an animal, such as a mammalian species (e.g., human) or avian (e.g., bird) species, or other organism, such as a plant. The subject can be a vertebrate, a mammal, a mouse, a primate, a simian, or a human. Animals may include, but are not limited to, farm animals, sport animals, or pets. A subject can be a healthy individual, an individual that has or is suspected of having a disease or a pre-disposition to the disease, or an individual that is in need of therapy or suspected of needing therapy. A subject can be a patient.

The “genome,” as used herein, generally refers to an entirety of an organism's hereditary information. A genome may be encoded either in deoxyribonucleic acid (DNA) or in ribonucleic acid (RNA). A genome may comprise coding regions that code for proteins or non-coding regions. A genome may comprise sequences of any or all chromosomes of an organism. For example, the human genome has a total of 46 chromosomes. The sequence of all of these chromosomes may collectively constitute a human genome.

The term “genetic variant,” as used herein, generally refers to an alteration, variant, or polymorphism in a nucleic acid sample or genome of a subject. Such alteration, variant, or polymorphism may be with respect to a reference genome, which may be a reference genome of the subject or other individual. Polymorphisms may comprise single nucleotide polymorphisms (SNPs). In some examples, one or more polymorphisms comprise one or more single nucleotide variations (SNVs), insertions or deletions (indels), repeats, small insertions, small deletions, small repeats, structural variant junctions, variable length tandem repeats, and/or flanking sequences. Genetic variants may comprise copy number variants (CNVs), transversions, or other types of rearrangements. A genomic alteration may comprise a base change, an insertion or deletion (indel), a substitution, a repeat, a copy number variation, or a transversion.

The term “polynucleotide,” as used herein, generally refers to a molecule comprising one or more nucleic acid subunits. A polynucleotide may comprise one or more subunits selected from adenosine (A), cytosine (C), guanine (G), thymine (I), and uracil (U), or variants thereof. A nucleotide may comprise A, C, G, T, U, or variants thereof. A nucleotide may comprise any subunit that can be incorporated into a nucleic acid strand. Such a subunit may comprise an A, C, G, T. U, or any other subunit that is specific to one or more complementary A, C, G, T, or U, or complementary to a purine (e.g., A, G, or a variant thereof) or a pyrimidine (e.g., C, T, or U, or a variant thereof). A subunit may enable individual nucleic acid bases or groups of bases (e.g., AA, TA, AT, GC, CG, CT, TC, GT, TG, AC, CA, or uracil-counterparts thereof) to be resolved. In some examples, a polynucleotide may comprise deoxyribonucleic acid (DNA), ribonucleic acid (RNA), or derivatives thereof. A polynucleotide may be single stranded or double stranded.

Systems and methods described herein may relate to genetic data management. Genetic data management may comprise to network architectures, reports, definitions and rules, directives and actions, storage devices and storage management, privacy, encryption, or compression.

Various types of sensors may be used to measure different genetic attributes. Some sensors may record and report different levels of resolution. Some sensors may provide native base sequence. In some cases, the sensors may detect chemical modifications such as methylation, amination/deamination, oxidation, and/or any other modifications and abasic (AP) sites in DNA and RNA.

The sensors may be configured to detect various types of signals, such as optical signals, electrical signals, or a combination thereof. Optical signals may include fluorescence, luminescence, chemiluminescence, bioluminescence, incandescence, lasers, light emitting diodes (LEDs), visible light, infrared radiation, near-infrared radiation, or combinations thereof. Electrical signals may include electrical current, voltage, differential impedance, tunneling current, resistance, capacitance, conductance, or combinations thereof. Some solutions for genetic detection may alter native molecules to detect them. Some detection methods, such as polymerase chain reaction (PCR), may rely on amplification, in which many copies of an original genetic polymer may be produced.

Amplification processes, in turn, may introduce apparent mutation errors that may render results inaccurate. Other error sources, such as electronic noise, phase errors, spectral deconvolution errors, fluidic diffusion errors, quantitation errors, position in a read, sequence context, spatial and spectral optical cross-talk, may also be present, which makes various sensors or detectors differ in terms of signal quality, types of error, measurement accuracy, or alternative interpretation of sensed or measured data.

In managing these different types of genetic data, it may be important to manage information about the source of the data, how they were measured, and the sensors, detection systems, hardware, consumables, chemistry methods, or software version used for measurement. Each set of data may comprise characteristic errors and uncertainties that may need to be accounted for in various situations.

Another issue in managing genetic data may be managing data storage. Different storage techniques and devices may be employed. Various types of specific storage media may be used, which may be designated in connection with a nature, quality, or quantity of the genetic data. Various types of genetic data, such as DNA or RNA sequences, may be stored in multi-cell storage devices. Blocks of memory may be used in various ways with respect to characteristics of the genetic data. For example, there may be a relationship between a size of a memory block and a type and size of data stored in the memory block.

Data Collection

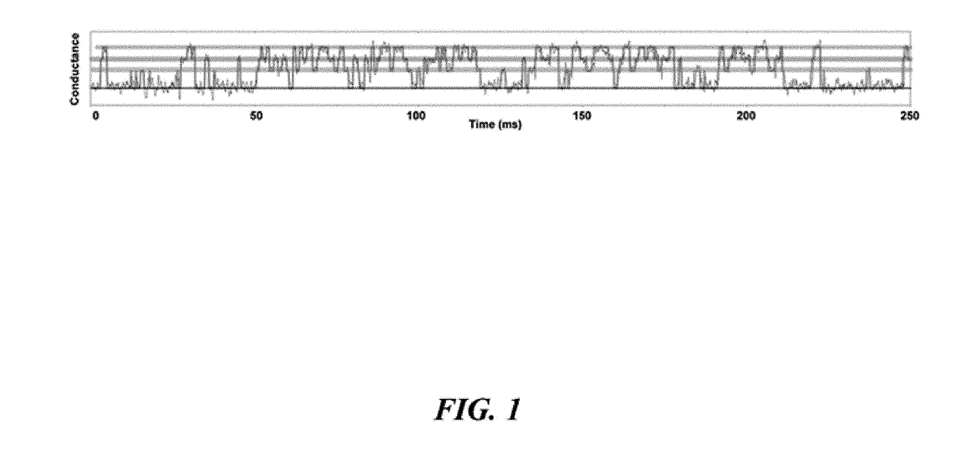

One or more biological sensors may detect raw data of molecular chains. Each raw data read may be converted into a native formatted record of the read. For example, if a sensor senses and measures electrical conductance, the sensor may produce a time series of conductance over time as a chain passes through the sensor, as shown in FIG. 1.

Conductance raw data may be later interpreted into nucleotide base data or records in the case of deoxyribonucleic acid (DNA) or ribonucleic acid (RNA).

Raw data from a sensor may be passed to an application server. Data may depend on a sensor type and may be derived from an electric property, such as conductance, capacitance, current (e.g., tunneling current), voltage, resistance, or any combination thereof. Data may comprise optical data, such as optical data derived from fluorescence (e.g., chemifluorescence) or absorbance, such as by fluorescent label tagging or modification of subunits (e.g., nucleic acid bases).

Transfer of data from a sensor to an application server may be performed using a wireless module integrated with a sensor through a wireless protocol, such as wireless fidelity (Wi-Fi), Bluetooth, or near field communication (NFC). Transfer of data may be performed using a wired connection, such as universal serial bus (USB).

The application server may comprise a desktop computer, a laptop computer, or a mobile device such as a mobile phone (e.g., iPhone or Android phone) or a tablet (e.g., iPad or Android tablet).

The application server may have instruction sets that receive the raw signal data and produce base data using certain base-calling routines. These routines may be programmed and updated on the application server based on the capabilities and characteristics of the sensor or other global directives, as described elsewhere herein.

The sensor updates can be received or pushed from the sensor manufacturer, for instance, to improve signal measurement or to alter hardware or firmware.

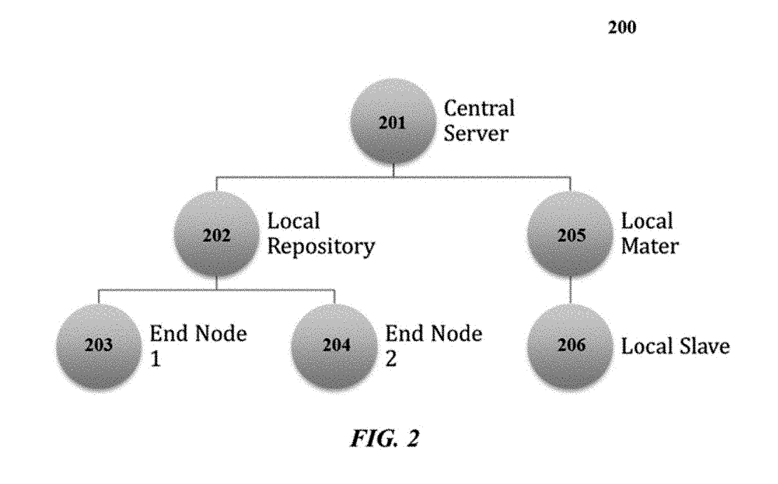

As shown in FIG. 2, an application server, or central server 201, may comprise, or have access to, a dedicated database of definitions and rules that the application server or central receives from a local repository 202. The definitions and rules may be updated as needed. The definitions and rules may identify various situations and actions. For instance, there may be pathogen signatures or sequences or any other data associated with a specific pathogen that may be detected by the local sensor. As such, the definitions and rules may be custom-made and may be dynamic. The application server 201 may be in communication with a local master 205, which may serve as a resource for data that cannot be interpreted or concluded by the application server. The local master 205 may be in communication with a local slave 206, which may stay in the same facility but may serve a limited function with quick access to the local master. The local repository 202 may be in communication with end node 1 203 and end node 2 204, which may be measurement devices.

As an application server performs a measurement, it may compare its results with definitions and rules it has access to, and may subsequently suggest directives accordingly.

If no definitions or rules are available for a particular situation, the application server may communicate this situation with its local repository 202.

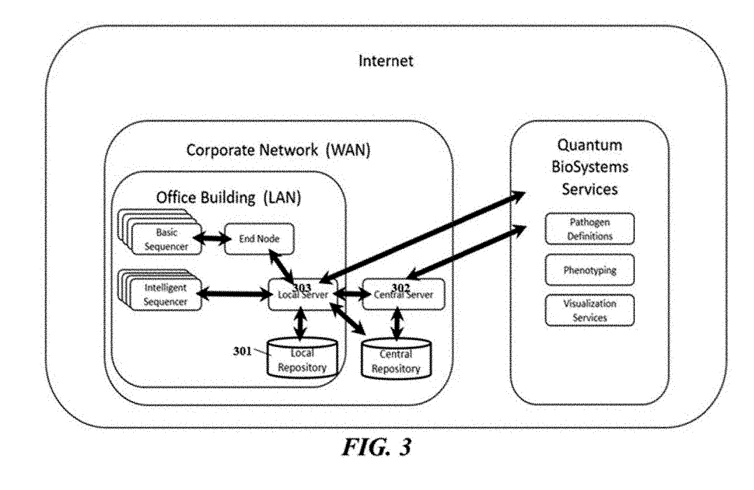

A local repository may comprise a server that is in network connection with one or more application servers, as shown in FIG. 3. The local repository 301 may comprise, or may have access to, a larger database and more definitions and rules, or more updated ones.

For example, the local repository may be in network connection with a central server 302. The central server may be in network connection with a number of local repositories 302 which may in turn be in network connections with local application servers 303.

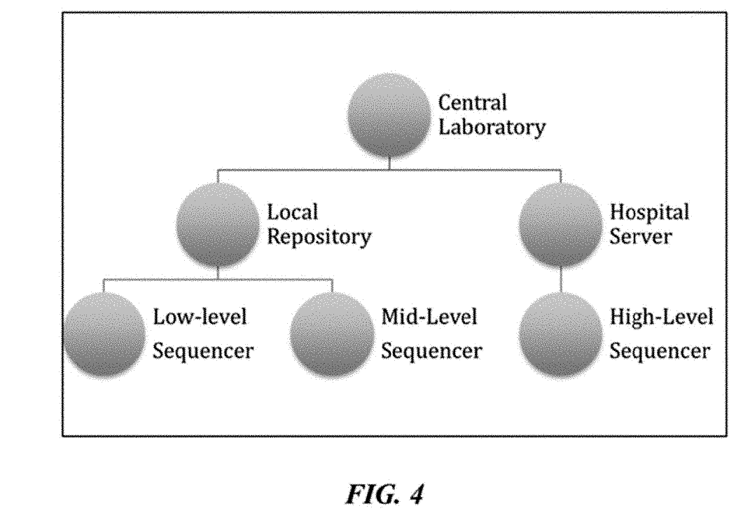

As illustrated in FIG. 4, the central server may be located at a central location, such as a national laboratory or a health organization facility.

A role of the central server may comprise communicating or updating definitions and rules along with directives to a number of local repositories or receiving reports from them.

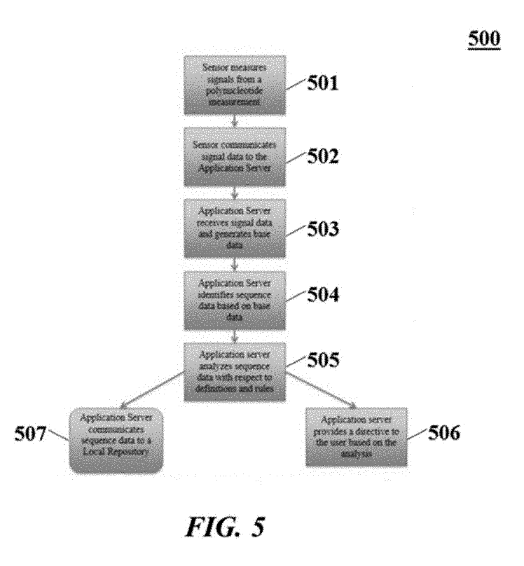

There may be several scenarios depending on the viewpoint from a certain machine. In some instances, one or more operations as shown in FIG. 5 may be performed with respect to the application server:

Sensor measures signals from a polynucleotide measurement 501;

Sensor communicates signal data to the application server 502;

Application server receives signal data and generates base data 503;

Application server identifies sequence data based on base data 504;

Application server analyzes sequence data with respect to definitions and rules received from a local repository 505;

Application server provides a message to the user based on the analysis 506;

Application server communicates sequence data to a local repository 507, if needed.

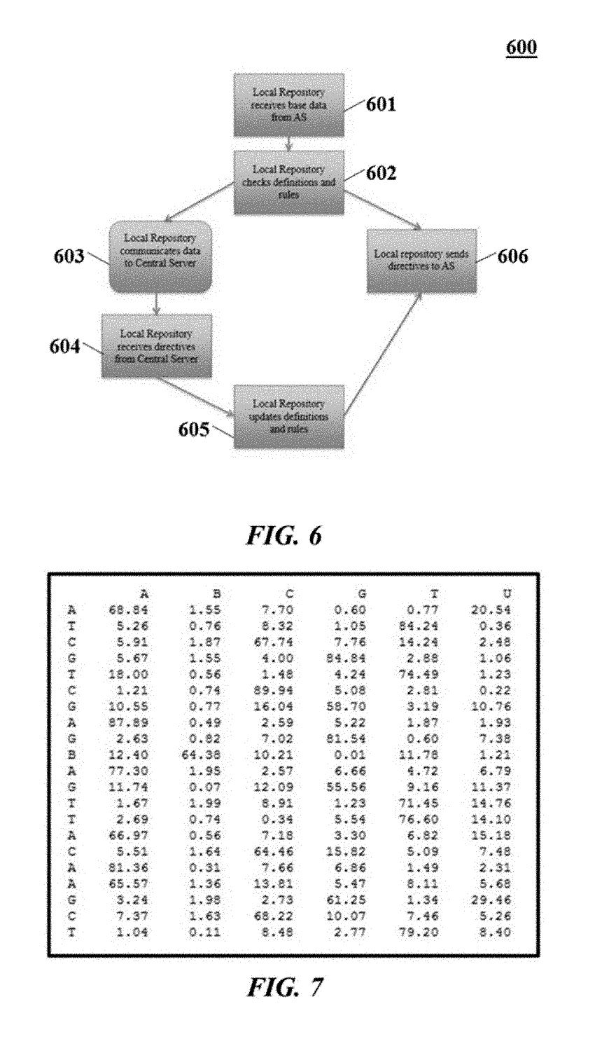

FIG. 6 illustrates possible operations performed by a local repository that may correspond to the set of operations described in FIG. 5 when an application server communicates sequence data to a local repository:

Local repository receives base data from the application server 601;

Local repository checks definitions and rules 602;

Local repository communicates abnormalities related to the base data to the central server 603;

Local repository receives global and regional updates from a central server 604;

Local repository updates definitions and rules 605;

Local repository communicates with Application Server new definitions and rules 606;

Central server communicates directives to the local repository; and

Local repository communicates directives to the application server.

The application server may be in direct or network communication with the local repository. The local repository may periodically send updates to the application server that the local repository has received from the central server.

The central server may be located at a central laboratory or a health center, and may analyze sequence data communicated by the local repositories. The central server may have access to a database of sequences.

### Example: Pathogens

A database of sequences may comprise a database of pathogen sequences. The central server may have faster access to recent pathogen sequences reported by using a faster memory and communication pipeline.

When a local repository receives information that may relate to a possibility of a new pathogen or a harmful known pathogen, the local repository may look for definitions and rules provided by the central server that may be related to the received sequence in a dedicated database. Based on a comparison of the received sequence data with sequences in the dedicated database with specific definitions and rules, the local repository may take appropriate options accordingly. For instance, the local repository may find specific rules and then pass specific directives to the application server.

Alternatively, if the local repository's definitions and rules meet a certain set of criteria, it may communicate the received sequence to the central server.

The central server may have access to a larger database, such as a comprehensive central database of recent and/or older breakouts. The central server may continuously update the central database based on what the central server collects from a plurality of local repositories.

The central server may be accessed by a central laboratory or a health center, where health or safety professionals have access and are alerted about events with specific predetermined thresholds.

Various decisions may be made by an authority running the central server. These decisions may comprise automatic or semi-automatic decisions. For instance, if the central lab determines that a certain sequence is not dangerous, the central lab may communicate to the local repositories a decision to ignore such instances. Alternatively, if there is an indication of a more serious situation, the central server may add the flagged sequence to a directive dedicated to such instances and keep the directive for faster access in a memory. Some subsequent instances reported to the central laboratory with a same or similar pattern may receive the same directive. The directive may comprise a decision regarding a medication, a quarantine, a rest, etc.

When a central lab has addressed and categorized a situation, the central lab may then establish definition and rules related to the situation. These definitions and rules and directives may then be communicated to local repositories of relevance. For instance, if a geographic outbreak is concluded, the central server may update any or all of the local repositories that are in connections to end users and application servers related to the area, while putting other areas in a vicinity of the area on alert.

In relation to food safety, a plurality of sensors in different locations may measure sequences from various types of food. The sensors at these locations may measure sequences and may search for pathogen candidates. Each sensor may be in communication with an application server. A sensor may measure signals from a sequence and send raw data to the application server.

The application server may comprise a set of definitions and rules. When the application server receives raw data from a sensor, the application server may run a program to produce base reads from the raw data and sequence contigs from the base reads. After the sequence contigs have been produced, the application server may run a program that compares the base data or sequence data with pre-established definitions and rules. These definitions may be in a database that the application server has access to. The definitions may be stored remotely on a dedicated server. There may be a subset of definitions that are designated as particularly important or crucial. For example, there may be a set of recent or current pathogen information. These particularly important or crucial data may be stored in a faster access memory or storage that the application server may have access to readily. In some situations, the application server may be instructed by a directive or a rule to search for a specific pattern. For example, this specific pattern may be related to current breakouts or reports from other sensors that may have indicated a pathogen in a similar type of food (e.g., produce).

The application server may be in network communication with a local repository. A local repository may serve a number of application servers with definitions and rules and may provide directives to the application server. The local repository therefore may periodically sends updates to the application servers.

If an application server does not find a proper definition or rule for a specific case, the application server may send the sequence data or other biological data to the local repository. The local repository may then search a broader database to which it may have access for definitions or rules. This database may be shared amongst one or more local repositories. The database may have a larger collection of known pathogens, for example, or may have some pathogens related to historical outbreaks that have not been observed for some period of time. Alternatively, such pathogens may not have been observed in a vicinity of the sensor location but the local repository may have access to a database that records the pathogens and therefore may be aware of them.

In special cases, the local repository may take any of multiple options. For instance, the local repository may look up definitions and rules related to the pathogen and communicate it along with certain directives to the application server. Alternatively, the local repository may communicate the data to a central server.

A local repository can have its own definition and rules which it receives from a central server. A central server can be in network communications with a number of local repositories. Accordingly, the central server can update definitions and rules at a local repository on a regular basis.

If a local repository cannot find any definition or rules for a particular case, the local repository may opt to communicate the data to a central server. A rule may require the local repository to report any base data, sequence data, or biological data that may indicate a special case.

A central repository may be located in, used in, or used by a central laboratory comprising researchers or health professionals. For instance, a national or international health center may be in control of the central repository. When a special case has been detected and communicated from a sensor to the central server, the central server may have access to a large set of definitions or rules to handle the situations. Optionally, upon reaching certain predetermined thresholds or at user discretion, researchers or health professionals may assess a situation to determine a severity of the situation.

A single sample may produce a plurality of gigabytes of raw analog conductance information representing millions of reads of sequence information. The initial interpretation process may consume these analog readings and may filter out background noise when no molecules are passing through the molecular sensors or when contaminants are causing unreliable or invalid results. The interpretation process may interpret and translate data into base sequence strings. Each base determination may be associated with one or more dimensions of data. For example, a dimension, or vector, may indicate a probability rating for what base it is reading, as shown in FIG. 7.

FIG. 7 shows a base probability matrix for a 21-mer reading by a sensor capable of sensing abasic (AP) sites or one of five possible bases. The determined base sequence 310 may represent a highest probability base at each location in the read. The possibilities of abasic sites or bases may comprise:

A=Adenine

B=abasic site

C=Cytosine

G=Guanine

T=Thymine

U=Uracil

Each column shows a probability of a specific nucleotide base at each location in the sequence. The sensor end node or an application server may interpret the probability for each possible base at each location. For example, this figure shows Cytosine (C) as the most probable base at the 16th base location.

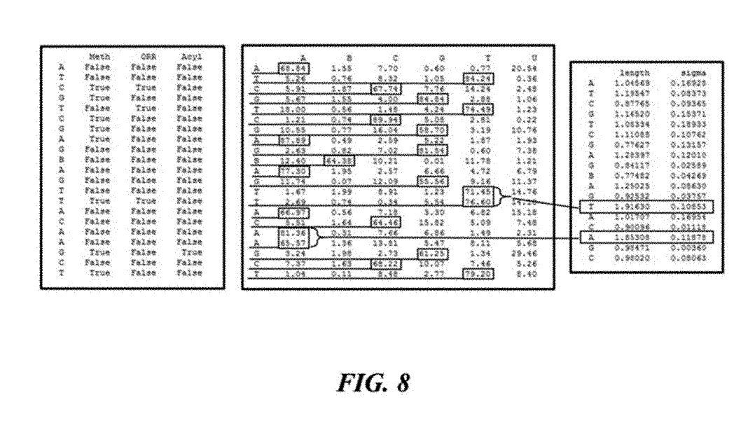

FIG. 8 illustrates how additional dimensions of data may be kept for a read. In this illustration, the modification table shows, at each base location, if the base is methylated, oxidized, or acylated. In this example, the third and fourth bases comprise a 5′-C-phosphate-G-3′ (CpG) pair that is methylated. The Cytosine (C) is also believed to be oxidized. The associated base probability table shows the determined base sequence. The distance table, or transition location table, contains the distances, in number of bases, between transitions to a new base giving the determined length of the homopolymers. The example shows a run of approximately two Thymine (T) bases before transitioning to an Adenine (A). It also shows two Adenine (A) bases before transitioning to a Guanine (G) later in the sequence. Storing dimensions of data for a read may address the type of sensor with intrinsic uncertainty regarding the number of same-type bases in a sequence or a sub-sequence.

Other dimensions may include an overall length and a base location as a distance from the beginning of the read. Some sequencing techniques start at one end of an oligonucleotide (oligo) and perform sequencing by synthesis (SBS). Such processes may involve looking for base incorporation after each round (e.g., one at a time). As such, there may be a possibility of generating phase errors each time a base is incorporated. For instance, if there is a clonal population, incorporation of the bases may be non-uniform across the population. Certain members may incorporate more than one base, while others may not incorporate a base. As such, confidence may decrease farther along the sequence read. A fourth dimension may incorporate a distance, in number of bases, base paired ends, or base transitions from the primer cleaved end of a sequence being analyzed.

Raw data reads may be kept for further analysis. For example, one may want to improve sensitivity by detecting polymeric creep, phototoxicity, a presence of contaminants affecting the sensors, or atomic structural changes to tips of nano gateways. The uncertainty in base call may be specific to the make and model of sensor used.

For instance, the interpretation process controller may pass each filtered conductance recording to a single interpretation worker process or thread. Each raw reading may be interpreted without concern for locking, since there may be no shared data. Synchronization may be unnecessary, since the processes downstream of interpretation may execute multiple times on the growing interpreted sample data set until the interpretation reaches its finished state with an acceptable degree of confidence.

Further, the system may incorporate sensors from different vendors to use various technologies to sense a sequence. In some cases, the raw information may not available. Instead, reads may be available from the sample where the probabilities and induced errors are specific to the technologies used. Each technology may have strengths and weaknesses, and may have various levels of sensitivity. Each technology may have various resolutions to various aspects or dimensions of reading DNA or RNA sequences. Some technologies may be highly sensitive to transitioning from one base to the next, but less sensitive to a particular base of interest. In this case, it may be desirable to conduct further analysis on the base reads.

Some technologies may be particularly good at base determinations, but less strong at determining base movement or transition. This situation may result in a high probability that it is looking at a particular base, but provide less certainty regarding the number of bases and when they repeat. Yet another technology may read each base along an oligo (e.g., one at a time) with an additive error model, such that the farther away from the starting marker, the less certain of the base being sensed.

Hence, various embodiments support interpreting sequence base data in various styles and formats for files and records when stored in non-volatile memory. For example, the data from a sample in an eXtensible Markup Language (XML) or JavaScript Object Notation (JSON) file may be stored on a distributed file system.

The file may comprise reads stored as a single base value for each nucleotide in the chain. The reads may be stored as a probability value. Alternatively, the reads may be stored as a complete probability matrix for each possible base at each nucleotide location. A possible syntax may comprise using one or more attributes to describe the meta-data syntax for what is stored in the read record.

There are various examples of semi-structured read formats with which various embodiments are capable of interpreting and working with, based on various factors involved in collecting the sample. Examples of such factors may include sample preparation, make and/or model of the sensors, or analysis of the data. Sample files may comprise a simple and basic schema comprising a unique sample identifier with one or more base reads.

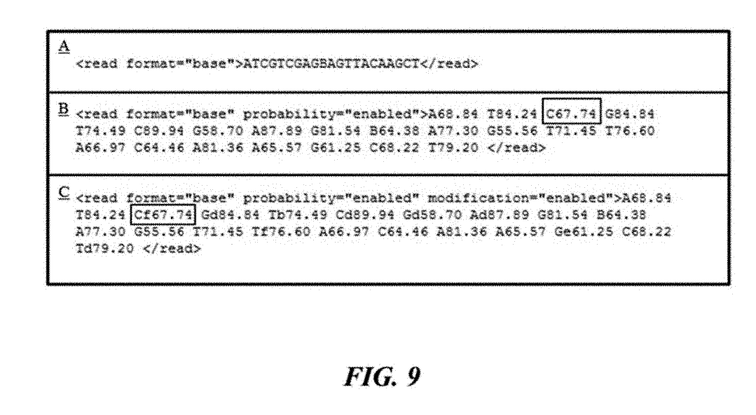

FIG. 9 shows examples of a sequence read, a base format read, and syntax. Part A shows a read comprising the determined base sequence. Part B shows an example of the same base format read including probability data for each base. The syntax for this second example comprises each word describing a single base. For example, the word “C67.74” describes the third base as a Cytosine (C) with a probability of over 67%.

The third example, shown in Part C, shows the same base format read with each word describing a single base location. In this example, each word describes a base, a probability, and any modifications. For example, the word “Cf67.74” describes the third base as Cytosine (C) with a 67% probability. Modifications may be recorded into each word by adding a lower case letter after the base. In this example, a lack of following lower case letters indicates that the base was not methylated, oxidized, nor acylated. The lower case letters “a” through “h” can be translated into the numbers 1 through 8 to hold a bit mask of the modification table. Methylation equals the most significant bit (MSB) (4), oxidation is (2), and acylation is the least significant bit (LSB) (1). Hence the Cytosine (C) base, modified by “f”, shows the Cytosine was methylated and oxidized.

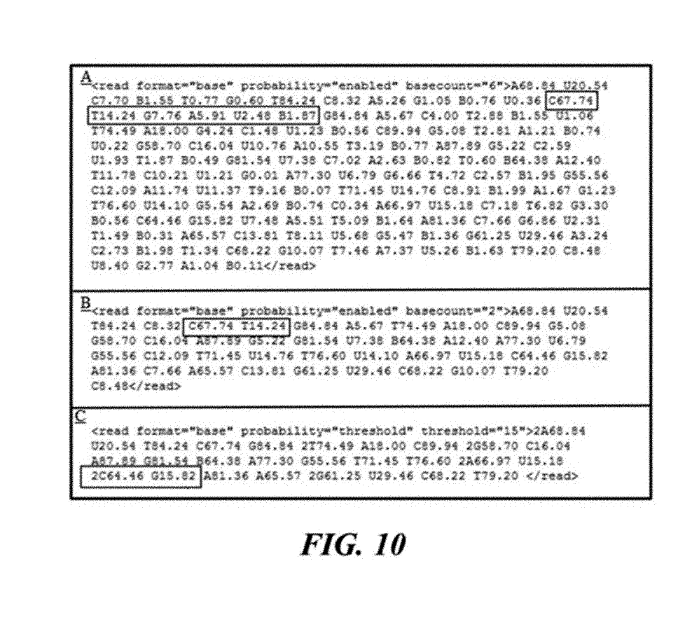

In accordance with the systems and methods described herein, it is possible to maintain secondary and tertiary possible base values, any modifications to those bases, and any other sensor-recorded dimension of data. FIG. 10 represents three examples of syntax for storing (A) each of six tracked base or AP site possibilities; (B) the highest two most probable bases or AP site possibilities; or (C) only maintaining an array of base location probabilities if the probability exceeds a certain predetermined threshold. In the first example shown in Part A, the file stores probabilities for each of the six bases and probability values for the third base location in the read as cytosine (C) having the highest probability at over 67% and an abasic site having the lowest probability at under 2%. If only the two highest probable base values are maintained, that base location may be seen as a primary cytosine (C) base and alternatively a thymine (T) base with a probability of approximately 14%, as shown in Part B.

Storing probabilities only if they exceed a predetermined threshold may be accomplished with a length/value syntax, shown in Part C. A base location with two base possibilities that exceed the threshold of 15% may result in a lead number “2” as the first character of the word “2C64.46”, which also provides the length of the array of bases kept for that base location. Cytosine (C) is the highest probability at 64%, and guanine also exceeds the threshold at 15%.

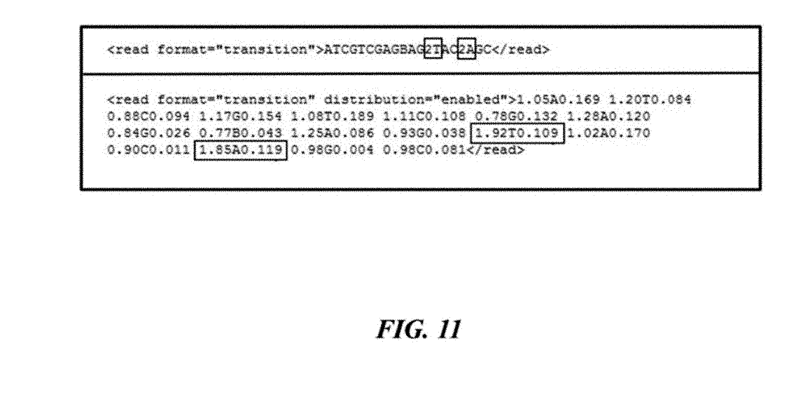

A transitional syntax for sensors that record a distance dimension between base transitions may also be used, as shown in FIG. 11.

The application server may collect millions of reads from a sample. It may then identify longer aligned sequence, or contig, data from analysis of the reads. For further evaluation, the application server may perform an alignment of the base reads against a reference. Alternatively, the reads may be grouped with several other reads and used in a de novo assembly. The application server may be extensible such that it may call other processes that accept only a subset of the information stored in the semi-structured format of the reads. For example, the interface to the alignment processes may accept a FASTA formatted syntax or a FASTQ formatted syntax for the reads. In this situation, the read may be translated into a format understood by the alignment processes.

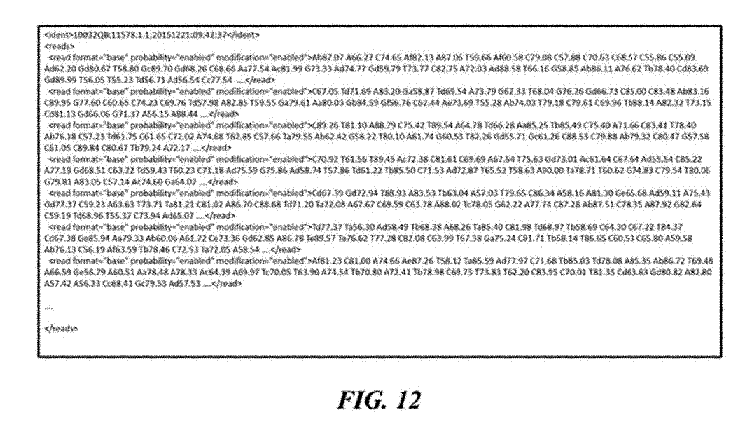

For instance, the example read described in FIG. 12, when translated into a FASTQ format, may look similar to the following four lines:

@10032QB:11578:1.1:20151221:09:42:37

ATCGTCGAGBAGTTACAAGCT

+10032QB:11578:1.1:20151221:09:42:37

‘*&*′+%+)&(%’(&&)&&&(

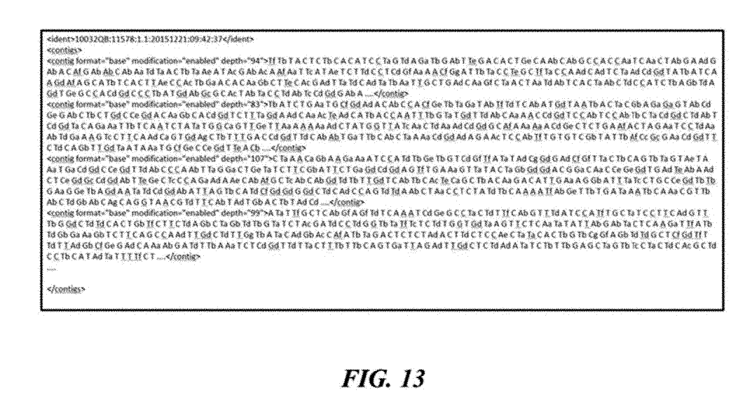

The bases and a corresponding Phread quality score may be sent. The reads may be interpreted and contigs may be returned from the consensus algorithms of the alignment processes. A sample may contain millions of reads. Reads may be either aligned against a reference sequence or assembled de novo. This translation of base reads into a different syntax may lose some context or resolution of the base reads. In an example shown in FIG. 13, the indicated sensors are able to capture transition distances and chemical modifications in addition to the base sequence and probability or quality score sent and returned by the programs that align the reads into contigs. The application server may take the alignments and, when the consensus is determined, reapply some lost context or resolution back into the sequence contigs, such that the contigs are stored in a similar semi-structured syntax as the reads. For example, for a contig derived from base reads that contain chemical modifications, the application server may reapply any modifications not used to sequence the reads.

The application server may analyze sequence contig data with respect to definitions and rules received from a local repository. An installation may be distributed with end nodes, servers, and/or repositories that are networked and cooperating to manage and act upon sequence data acquisition. In an aspect, the application server may incorporate rules to discover and act upon genetic sequence information with high efficiency. Sequence discovery may be directed to find a pathogen. In other cases, one may want to discover contigs for certain gene expressions. Various embodiments allow one, such as a microbiologist, to administer a database of sequence definitions for the pathogens or genes. Rule definitions may be assigned to, or associated with, a specific directive or set of directives.

The central controls and rules management module may process these rules. In some cases, they may translate the rule or further modify it, such that it runs on specific downstream servers and nodes. Many rules will be distributed themselves.

For example, a rule may comprise a simple sequence, a matching method, a weighting, one or more regression adjustments, or directives to bundle the sample information into a National Center for Biotechnology (NCBI) compliant BioSample and to notify a department head.

The instantiation of the system in this example may include a basic sensor, a local node, and/or a local server. Rules may be adjusted to a specific piece of equipment where it executes. An application server may attempt to discover a sequence from each individual read or contigs. The discover portion of the rule may be better served by modifying the higher level rule to more effectively discover the sequence based on a make or a model of the sensor used. The rule at a high level may be to align a sequence to a contig with less than a predetermined number of variances based on the type of sequencing equipment used. In some cases, a global method and valuation may be used, while with other sequencing equipment a local method and valuation may be applied. Alternatively, the sequence to contig mapping may have a threshold variance level based on a flowgram, e.g. if the sensor used was a Roche 454.

In an embodiment, rules may be distributed and may comprise cooperation with dedicated application servers. This may allow for more accurate results with fewer false results without adversely affecting overall performance of the end sequencing equipment. For example, an installation may have a plurality of sensor nodes testing food samples:

These read signals are sent to an application server for interpretation into base reads and subsequently contigs.

This initial application server executes a rule with a simple lower processing cost sequence alignment algorithm on each base read against an array of pathogen signatures.

If a threshold for a number of close matches or score is met for one or more of the pathogens, then the directive may include:
extending the sampling at the sensor; and/or

bundling the complete sample and forwarding it to a dedicated pathogen testing application server for a more rigorous interpretation of the sensor measurements.

The pathogen testing application server may then apply its own directives based upon its findings.

This embodiment may ensure the information is protected, both when the information is being communicated across networks and when the information is stored in a repository.

For data in transit, encryption schemes such as secure socket layer (SSL) or transport layer security (TLS) may be applied. Data may be produced at the sensors. These end node sensors may support connections to local application servers, which analyze the raw data into base reads. The application server may further analyze the base reads into contigs or sequences. Alternatively, the application server may communicate the reads to another application server to create the base reads and sequences. Communications between sensors and application servers, between cooperating application servers, between application servers and repositories, and between application servers and services may support secure sockets layer (SSL) or transport layer security (TLS) connections. This may include servers that associate base reads and sequences with other meta data, such as names or geographic locations, and apply rules and directives.

For data at rest (e.g., not in transit), various mechanisms may be used to protect the data. Data may be stored in a plurality of locations. Sample data may be stored in a file system. Each sample may comprise a semi-structured data file. A process may perform marshalling, unmarshalling, and/or removal of sample files.

Derived contig or sequence data may be stored in a similar way as a plurality of semi-structured files. Contig data may be kept in a distributed file system, since the contig data may comprise a large data set, may be continuously mined and analyzed to test hypotheses, and may require a repository that can support access with high parallelism. As with sample files, a process may perform marshalling, unmarshalling, and/or removal of contig files. These files may be anonymized. The encryption and compression mechanisms may be tuned for lower central processing unit (CPU) costs of access and higher throughput in reading.

When sequences are stored into a repository, only an identifier may be associated with the contigs. They may be de-identified with respect to the subject, location, contact information, or study corresponding to the sample. The identity data may be stored in a separate repository from the sequence. Likewise, base reads from samples may be associated only with an unique identifier. If raw data is retained, it too may only be associated with an identifier. Identity data may be placed in a separate database. The identity data may be kept in a relational database. A sample-identity and contig-identity reference table may be maintained to allow the linkage to re-identify a pair of a sample and a contig if access controls allow. A different set of access controls may be applied to the anonymized samples. Both the identity data and the sequence data may be encrypted at rest.

Sample data, contigs, and sequences may represent relatively static data sets. Upon being added to a repository, they may be seldom updated. They may represent as much as petabytes (e.g., millions of gigabytes) of data. Analytical processing of these extremely large data sets may be enabled through the use of a distributed file system storing protected semi-structured data sets that may be accessed and reduced through processes, such as MapReduce or Spark, into working transactional or columnar databases.

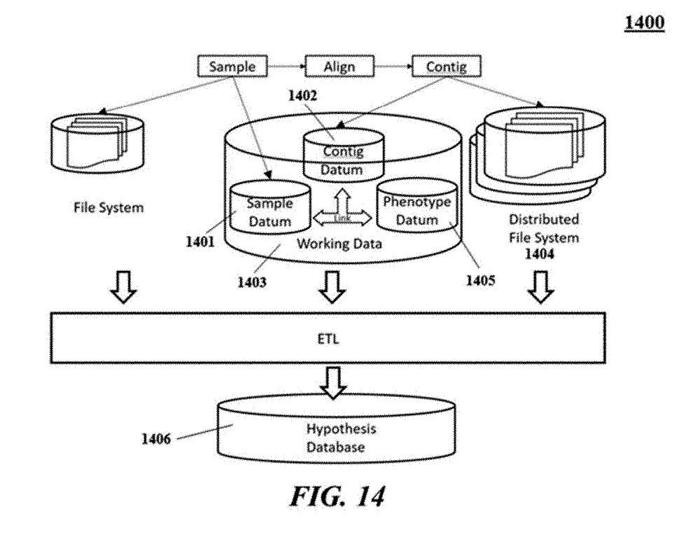

For instance, FIG. 14 illustrates an example of a distributed file system where the information is retained in three separate storage systems— one each for samples 1401, contigs 1402, and working data 1403. Raw sample data 1401 may be interpreted and translated into a semi-structured format consisting of the molecular reads along with simple or basic meta-data concerning the sample. The basic meta-data may comprise a sample identifier. All other meta-data regarding the sample may be considered working information. Working information may be stored separately in a database with a reference to the sample identifier. Once processed, sample data may or may not be retained. If sample data is retained for long periods of time and is used or accessed for other purposes, it may be stored in a distributed file repository 1404. Alternatively, if sample data is retained for long periods of time but is not commonly accessed and used for other purposes, it may be archived.

Sample data may be further interpreted, aligned, or assembled into sets of contigs or sequences. These contigs may be stored in a distributed file system 1404, in a semi-structured format, such as XML or JSON, with an assigned a contig identifier. In a similar manner as sample data, other meta-data regarding the contig may be working information and may be stored separately in a database with a reference to the contig identifier.

Contigs also may have working data. Working data may comprise additional data captured and used other than the reads and derived contigs. This may include information regarding the process involved in capturing the information, such as a make, model, or serial number of the equipment used; sample preparation information; source information; a location at which the sample was obtained; and protected health information such as names and contact information of a patient.

These sample data and contig data files may be compressed to increase capacity, with the understanding that in doing so, there is a computational cost incurred when reading the files. These files may be encrypted. As the information within these files may be anonymous, an embodiment uses an encryption algorithm that employs a highperformant (e.g., secure) decrypting counterpart. Hardware cryptographic accelerators may be employed to minimize encryption and decryption costs.

Working data may comprise additional information stored in order to re-identify or work with samples and contigs. The working data also may include a phenotype schema with associations between identities, sequences, and phenotypes 1405. Working data also may be encrypted. However, whereas performance may be an important factor in deciding which algorithms to use, security may be an important factor for the working data. Further, fine grain security and access, such as record-level access, may be implemented for working data.

The sample storage and the contig/sequence distributed storage may encrypt the semi-structured files using a symmetric key. Application server processes responsible for marshalling and unmarshalling the files may maintain a list of ciphers for files in a secure wallet. Additionally, hosts upon which the application server processes are running may include an accelerator, such as an Intel Advanced Encryption Standard-New Instructions (AES-NI).

Among the benefits of the embodiment may be that the repository is modeled to maintain and provide necessary tools to access and mine a large collection of bioinformatic information that the repository is capable of storing over a long period of time in an anonymous context. The anonymous contigs and optionally initial sample data may be retained and may be securely made available to researchers in improving understanding of genetics.

In some embodiments, a physician may be able to access a patient medical record comprising both the genetic contigs linked to the associated working information. In this example, the physician is within an application that provides two different types of accesses: a performant access to specific contig and sequence sets and a secure access to the working data linked to the contigs and sequences.

### Example 1: Research

In research contexts, raw data of samples from a plurality of sensors of various manufacturers are sent to an application server. The application server interprets the raw data and determines the base sequences of a portion of or all of the reads in the raw data. The application server then either performs the alignment analysis itself or formats the reads into a syntax understood by an external alignment analysis server tool to which it calls out. The resulting contigs are returned from the external server to the application server.

In some cases, the application server re-applies information from the sample reads back into the contigs. The re-constituted contigs are tagged with an identifier and transmitted to the contig repository, where they are saved as semi-structured files in the application server's distributed file system. Additional information, such as source, identity, location, and/or address, related to the contigs are inserted into the repository's working database.

Additional meta information may be incorporated in the semi-structured files, such as taxonomy, to allow for efficient storage in the distributed file system or to reduce the data during an extraction. The repository of contigs grows over time.

A researcher hypothesizes on relationships between specific genetic signatures and a cause or probability of some expression of one or more phenotypes. The contig repository is mined. Specific signatures and their associated identifier are extracted as independent variables and loaded into a database for testing the researcher's theory.

Signatures may then be mapped to phenotypes obtained from external sources.

Hypotheses that prove useful may be saved and incorporated into an application server in a separate database 1406 of gene signature associations to gene expressions and phenotypes.

Semi-structured files are encrypted, as is the database. Access is controlled to the level of the sample and contig identifier.

Sample and contig information may be retrieved without working information with a different level of security. For example, a researcher may be allowed access to all the contigs in the system, but not to any contig with its associated working information.

Access control is abstracted and may support concepts such as group and role security. Fine-grain security with abstract controls provides effective security and privacy over time. As an example, employees of a medical group may access an embodiment that stores bioinformatic information on a portion of or all of the patient members of the medical group. Over time, the doctors responsible for a particular patient may change. Doctors may have access to only the bioinformatic information of patients for whom they are currently responsible.

Access is granted through strong public/private key management systems and provides support for nonrepudiation.

A management program may manage the nodes and users of the system. The management program may incorporate certificate authority services for issuing keys and maintaining the certificate revocation list. Processes running in the end node sensors, application servers, and distributed file system manager have public/private key pairs that allow them to act upon the information. Users also have generated key pairs. A user may have multiple key pairs associated to his account to support authentication from a plurality of different computers, tablets, or other computing devices.

The concept of roles or groups is supported. Accessing stored data is controlled by roles, while a currently active user may belong to one or more roles.

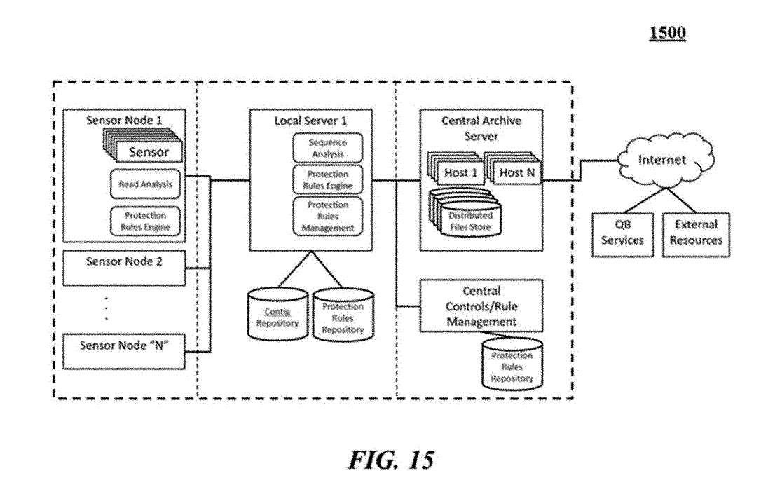

This architecture and abstraction of access controls for data at rest has the added benefits of ensuring a portion of or all sequence information is secured and made available only authorized entities over the life of the data records. FIG. 15 shows an exemplary architecture illustrating segmented access control.

Access control is capable of being fine grained, e.g., to the individual sample level. Each sample may be tagged with a unique identifier.

For jobs that are not crucial in nature, a low-level sequencer or biological sensor may be used. A low-level sequencer or biological sensor may not require a large permanent storage device. Examples of such a device may include measurement or data acquisition modules. Such a device may have measuring hardware, a processor, and/or a system memory for handling system functions. Each of these components may have its own buffer memory for handling its own functions.

A low-level sequencer may require a communication link to relay its raw data to higher-level device such as an application server, a local repository, or a local server.

The communication link may comprise a near-field communication protocol, such as Bluetooth or near field communication (NFC), or a wireless protocols such as Wi-Fi. The communication link may comprise a cabled (e.g., wired) communication provisions such as USB. In some cases, the communication link may comprise a satellite or a cellular communication module.

A low-level sequencer may be integrated with an application server that may be operating on a mobile device such as a mobile smartphone to perform some of these aforementioned functions. For instance, the low-level sequencer may comprise measurement hardware and use mobile device capabilities and applications as a local memory, processor, and communication link.

Alternatively, a mid-level sequencer may be used in more critical circumstances. Examples of such critical circumstances may include monitoring of patients and pointof-care applications where an initial diagnosis is needed.

A mid-level sequencer may perform more accurate measurements of a polynucleotide. The accuracy may be set according to what is needed for a reliable accurate judgment of a sequence.

A mid-level sequencer may use a memory device and a communication component. Hence, the mid-level sequencer may include measurement and data acquisition modules with measurement hardware, a processor, and a system memory for handling system functions. Each of these components may comprise its own buffer memory for handling its own functions.

The additional memory device may comprise a flash memory (e.g., multi-level cell flash memory) capable of storing bits of data. The data in a mid-level sequencer may be base data, in which case a multi-level cell flash memory may be suitable to store the data locally. A port such as a USB port may be used to transfer the data, e.g., in cases where there is a lot of data such that a wired connection may be desirable for high bandwidth or throughput purposes.

In an embodiment, a multi-level cell device such as a flash memory is used as a relatively fast way of storing and accessing genetic sequence data. In a flash memory storage device, a large number of cells may be used to store data based on floating gate field-effect transistors (FETs) that are capable of holding a charge. Cells may be programmed individually by charging the floating gate of each FET.

One advantage of this embodiment is due to the fact that flash memory cells may be erased in blocks, via block erase operations, thereby erasing all charge of all of a plurality of floating gates in a single operation.

This embodiment may also have a characteristic that individual cells are not eraseaddressable. However, in this embodiment, an erasable block of the flash memory is used to store genetic data related to a sequence of bases, nucleotides, or otherwise contiguous genetic data. In case one needs to replace this erasable block, a user may typically wish to erase all of the data in the erasable block at once, rather than a portion of the erasable block. This embodiment therefore may allow flexibility of optimizing cost versus speed for genetic data storage.

In a flash memory storage device, cells may start to fail after a number of program and erase cycles, after which point, reading or writing may fail. This fact can be used advantageously for genetic data storage. Since the number of erase cycles of a flash memory may be limited, the data may be kept safe for a longer time than some other usage scenarios.

There may be specific relationships between erase block size and sequence or otherwise genetic data size. This may ensure integrity of the data related to the whole sequence.

As a specific example, a sequence of bases consisting of 128 kilo base pairs (kbp) is stored in an erase block of 128 cells:

CTT . . . GAG (128 k bases)

=== . . . ===(128 k cell erase block)

For native DNA and RNA bases, a two-bit multi-level cell (MLC) may be dedicated to each base. For instance, for the case involving DNA, one uses:

A(00) C(01) G(10) T(11)

which means, both the first and the second bits are off when the base is an A, the second bit is on when the base is a C, the first bit is on when the base is G, and finally both the first and the second bits are on when a base is T. A similar scheme may be used for RNA.

Each erase block may be designed or configured to store multiple sequences. Alternatively, a larger sequence may be stored on a specific number of erase blocks with similar or same properties and life cycle.

Differently-sized erase blocks may be used for differently-sized sequences. For instance, flash memory devices of a smaller erase block size may be used to store oligo data or hybridization data, while flash memory devices of a larger erase block size may be used to store genes and mutations or reference genes. Flash memory devices of a large block size may be used to store genome data.

An advantage of using flash memory for faster access may be compromised by life cycle issues. A copy of flash memory content may be mirrored on a storage server with slower access but longer life cycle. A test may then be devised to probe the integrity of data in each block size. Occasionally the data in each block may be tested against the mirror data in the server. Should the flash memory erase block data show any sign of degradation, that block of the flash memory devise may be decommissioned.

This embodiment may be advantageous at least since the longer life cycle storage device may be, for instance, a remote hard disk drive (HDD) storage server in the cloud.

In a further example, an erase block of a flash memory storage device may be used to store sequence data plus some metadata:

CTT . . . GAG (96 k bases)-Metadata (64 k bit=32 k cell MLC)

=== . . . ===(128 k cell erase block)

Examples of metadata may include any information related to the origin of the sequence, such as name of a patient, other information related to a patient, or the sequence itself.

A shorthand of the biological data may optimize the size of the data with respect to the storage device architecture, for example, by using a compression or the biological data. The size of the compressed data may be fine-tuned for better storage device compatibility.

A hash table may be made of different biological data. Each hash may correspond to a one category or genre. For instance, in case of proliferation of pathogen data, one may build a hash for each pathogen and use a hash table. Whenever a new sample is measured, performing a hash of the new sample may readily find a match within the hash table. This is a fast and efficient way of obtaining information about the pathogen.

A multi-level cell (MLC) storage cell may store two bits. The two bits may be used to store information about a base of a polynucleotide. For example, for a DNA base, the following bit configurations may be used:

00 A

01C

10G

11T

In this way, all native four bases may be represented using a single memory cell. This approach may be advantageous for ensuring integrity of data.

In another example, an MLC storage cell may store three bits. The three bits may be used to store information about a base of a polynucleotide with additional information indicating methylation or oxidation status. For example, for a DNA base, the following bit configurations may be used:

### 000 Native A

### 001 Native C

### 010 Native G

### 011 Native T

### 100 Oxidated A

### 101 Methylated C

### 110 Abasic

111 Other information

In this manner, multi-cell memory devices such as flash memory and phase change memory may be used.

In case of data degradation in a storage device with blocks with multiple cells, loss of data may be avoided by providing a warning, by refresh cycles, or by automatic or instigated dumping of data into a storage server, e.g., a HDD, or into a cloud storage server.

Erase blocks in a flash memory device may be used for ease of access and storage management. When all the data on an erase block corresponds to a biological unit, for example, a DNA or RNA sequence, memory access may be economized and data may have more integrity. This may lead to power optimization in large-scale operations where many sequences areas or genetic data may be accessed and may be operated on in a short time.

Data integrity may be preserved through this embodiment by keeping all the data relevant to a certain genetic unit, such as a gene or a contig, in a certain unit or units of memory. In addition, other benefits such as processing, optimization, and reducing generated heat may be achieved. It is envisioned that data management, data compression, memory access, temperature control, and data integrity may have a positive net effect on the entire ecosystem of biological data management, whether local or global.

A memory block, such as a flash memory erase block, may be chosen to be compatible with the size of the genetic data. Toward this end, customized compression and variance analysis may be performed to make the compressed size of the genetic data more optimal to the size of a memory block or a memory bank. The optimization may be performed in terms of data loss and data preservation. For example, in case a memory unit size, such as a block size or bank size, is larger than the size of the biological unit data, the rest of the memory space may be used to store additional information about the biological unit data. For example, an erase block in a flash memory may be used to save gene information, while additional information about the gene, such as gene expressions, may be saved on the remaining space of the block.

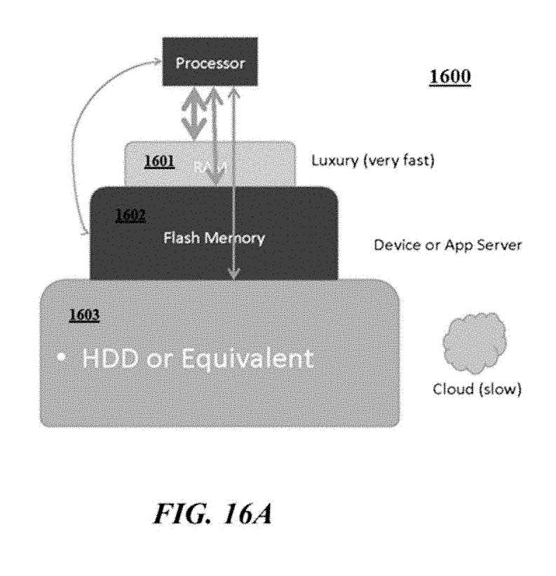

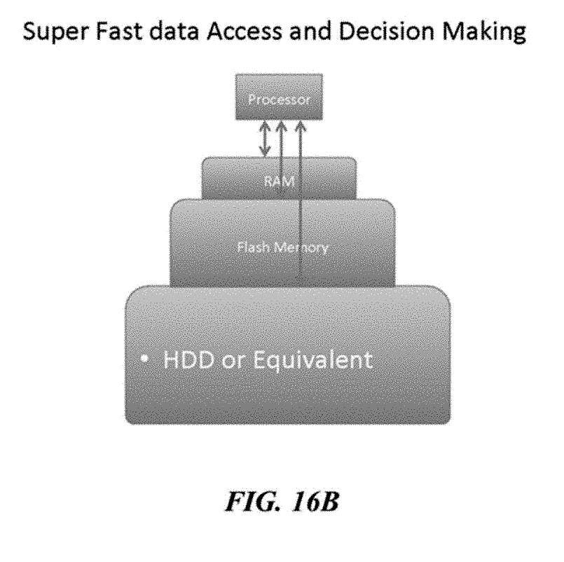

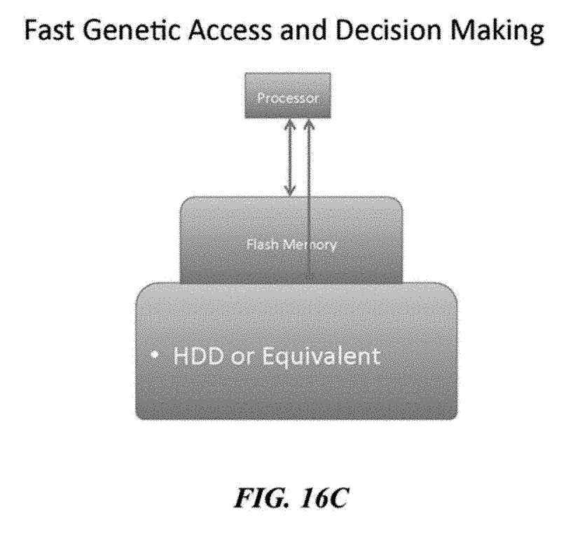

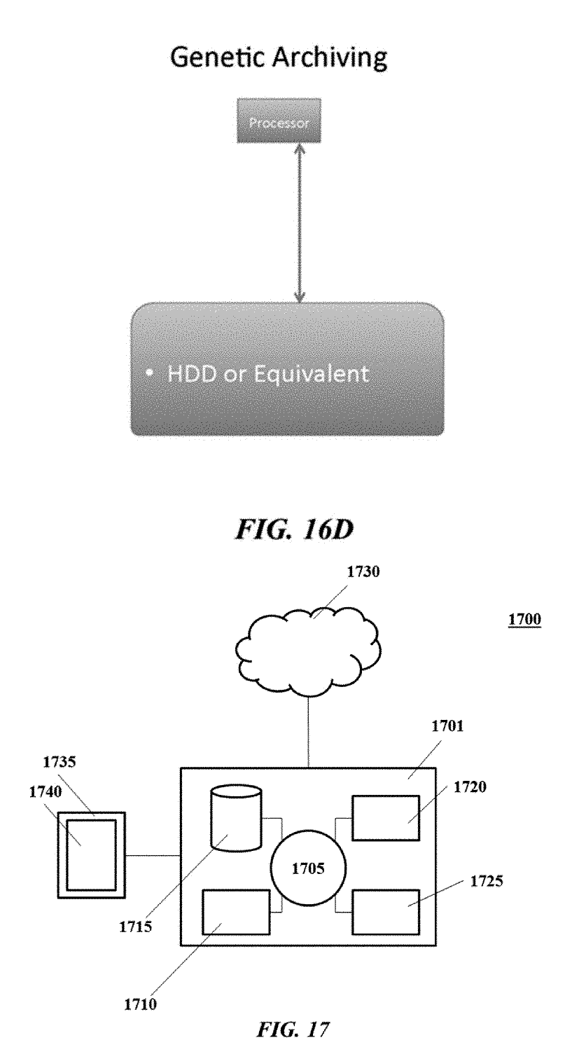

Access to biological data may be managed through a tiered storage access scheme, as shown in FIG. 16A. An application may be on a local repository or central server. First tier access may be achieved through using a fast memory. In crucial cases, a random access memory (RAM) 1601 may be used to access certain data that needs to be frequently accessed. In less crucial systems, the fast memory may comprise a flash memory 1602 in or adjacent to a local HDD or a cloud-based storage unit.

The decision to retain certain biological data may be based on a hit-or-miss architecture. When a certain number of hits are registered, a processor may access the biological data and may escalate it to faster memory (e.g., by copying or moving the biological data). For example, upon detecting a report of instances of a pathogen, a local repository or a central server may decide to bring a copy of the pathogen to local memory. Further upon identifying specific regions of the biological data unit that may be of importance, a copy of the specific region may be maintained in faster memory and the rest of the data unit may be kept at a lower level in a slower memory, for example HDD, cloud, or equivalent 1603. FIGS. 16B, 16C, and 16D provide additional examples of storage architectures. FIG. 16B shows an example of an architecture suitable for providing super fast data access and decision making, in which a processor can be configured to communicate with a RAM, a flash memory, and/or an HDD or equivalent. FIG. 16C shows an example of an architecture suitable for providing fast genetic access and decision making, in which a processor can be configured to communicate with a flash memory and/or an HDD or equivalent. FIG. 16D shows an example of an architecture suitable for providing genetic archiving, in which a processor can be configured to communicate with an HDD or equivalent.

### Example 2: Privacy Encryption

An example is provided of an encryption technique applied to genetic sequence data for an imaginary person by the name of Michael Smith and a 16-mer sequence related to him. The 16-mer may be a part of a larger sequence, gene, or genome related to the person.

Michael Smith-
. . . t t g c g a t g t c t a a t g g . . .
(subject sequence)

In this example, the name “Michael Smith” is encrypted using a 24-bit cypher for the purpose of illustration. The encrypted name and corresponding syntax are expressed as:
Encrfn(“Michael Smith”, cypher1)=EnCt2568e6c561c2b3a78926b5dbb3adea5ba827c065e568e6c561c2b3a78926b5dbbJ IGwNtmg( )ACHd+Q9e1ZHTMJV2DqVe3XSDb77IwEmS

This approach may ensure privacy of the name, as long as the cypher is secure. This type of encryption and subsequent decryption and cypher protection is potentially computationally intensive and costly. It may be appreciated that, in this example, the name of a person, which may be comprise few bytes, may grow by a few hundred bytes if extensive encryption is used.

To ensure privacy of the sequence, it may be assumed that there is reference sequence containing:

t t g c g a a g t c t a a t g g . . .
(reference sequence)

The bold and underlined base is assumed to be the only varied base in the population.

Then, it may be assumed the original sequence taken from Michael Smith contains the following:

. . . t t g c g a t g t c t a a t g g . . .
(subject sequence)

According this embodiment, this sequence is stored as:

. . . t t g c g a a* g t c t a a t g g . . .
(subject sequence representation)

where * may be a number from 0 to 3, thereby giving:

a0=a
a1=c
a2=g
and
a3=t

In the case of Michael Smith, this number is taken to be 3, shifting an “a” to a “t”.

This example shows that the sequence

. . . t t g c g a a(0123) g t c t a a t g g . . .

may represent the entire population with an expense of a two-bit character, in this case (0,1,2,3).

Since the rest of the sequence is identical for the entire population, according to this embodiment, complete privacy of the sequence may be achieved with an expense of a 2-bit key.

In this example, a portion of a oligo or contig is presented where only one base is variable compared to a reference oligo or contig.

In this example, to encrypt this sequence, the reference sequence is assumed plus a 2-bit code (123) that may shift one base by 1-3 places according to an encryption scheme, e.g.:

a c(1) g(2) t(3)

If the encrypted variable base was a “g”, for example, the shift function in the encryption code may give:

a(2) c(3) g t(1)

Similar schemes may be used without departing from the scope of this embodiment.

Computer Control Systems

The present disclosure provides computer control systems that are programmed to implement methods of the disclosure. FIG. 17 shows a computer system 1701 that is programmed or otherwise configured to manage biological data. The computer system 1701 can regulate various aspects of data management of the present disclosure, such as, for example, the collection, storage, encryption of biological data, communication between servers, servers and repositories with respect to definitions and rules, and management definitions and rules. The computer system 1701 can be an electronic device of a user or a computer system that is remotely located with respect to the electronic device. The electronic device can be a mobile electronic device.

The computer system 1701 includes a central processing unit (CPU, also “processor” and “computer processor” herein) 1705, which can be a single core or multi core processor, or a plurality of processors for parallel processing. The computer system 1701 also includes memory or memory location 1710 (e.g., random-access memory, read-only memory, flash memory), electronic storage unit 1715 (e.g., hard disk), communication interface 1720 (e.g., network adapter) for communicating with one or more other systems, and peripheral devices 1725, such as cache, other memory, data storage and/or electronic display adapters. The memory 1710, storage unit 1715, interface 1720 and peripheral devices 1725 are in communication with the CPU 1705 through a communication bus (solid lines), such as a motherboard. The storage unit 1715 can be a data storage unit (or data repository) for storing data. The computer system 1701 can be operatively coupled to a computer network (“network”) 1730 with the aid of the communication interface 1720. The network 1730 can be the Internet, an internet and/or extranet, or an intranet and/or extranet that is in communication with the Internet. The network 1730 in some cases is a telecommunication and/or data network. The network 1730 can include one or more computer servers, which can enable distributed computing, such as cloud computing. The network 1730, in some cases with the aid of the computer system 1701, can implement a peer-to-peer network, which may enable devices coupled to the computer system 1701 to behave as a client or a server.

The CPU 1705 can execute a sequence of machine-readable instructions, which can be embodied in a program or software. The instructions may be stored in a memory location, such as the memory 1710. The instructions can be directed to the CPU 1705, which can subsequently program or otherwise configure the CPU 1705 to implement methods of the present disclosure. Examples of operations performed by the CPU 1705 can include fetch, decode, execute, and writeback.

The CPU 1705 can be part of a circuit, such as an integrated circuit. One or more other components of the system 1701 can be included in the circuit. In some cases, the circuit is an application specific integrated circuit (ASIC).

The storage unit 1715 can store files, such as drivers, libraries and saved programs. The storage unit 1715 can store user data, e.g., user preferences and user programs. The computer system 1701 in some cases can include one or more additional data storage units that are external to the computer system 1701, such as located on a remote server that is in communication with the computer system 1701 through an intranet or the Internet.

The computer system 1701 can communicate with one or more remote computer systems through the network 1730. For instance, the computer system 1701 can communicate with a remote computer system of a user (e.g., a laboratory or hospital). Examples of remote computer systems include personal computers (e.g., portable PC), slate or tablet PC's (e.g., Apple (Registered trademark) iPad, Samsung (Registered trademark) Galaxy Tab), telephones, Smart phones (e.g., Apple (Registered trademark) iPhone, Android-enabled device, Blackberry (Registered trademark)), or personal digital assistants. The user can access the computer system 1701 via the network 1730.

Methods as described herein can be implemented by way of machine (e.g., computer processor) executable code stored on an electronic storage location of the computer system 1701, such as, for example, on the memory 1710 or electronic storage unit 1715. The machine executable or machine readable code can be provided in the form of software. During use, the code can be executed by the processor 1705. In some cases, the code can be retrieved from the storage unit 1715 and stored on the memory 1710 for ready access by the processor 1705. In some situations, the electronic storage unit 1715 can be precluded, and machine-executable instructions are stored on memory 1710.

The code can be pre-compiled and configured for use with a machine having a processer adapted to execute the code, or can be compiled during runtime. The code can be supplied in a programming language that can be selected to enable the code to execute in a pre-compiled or as-compiled fashion.

Aspects of the systems and methods provided herein, such as the computer system 1701, can be embodied in programming. Various aspects of the technology may be thought of as “products” or “articles of manufacture” typically in the form of machine (or processor) executable code and/or associated data that is carried on or embodied in a type of machine readable medium. Machine-executable code can be stored on an electronic storage unit, such as memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links or the like, also may be considered as media bearing the software. As used herein, unless restricted to non-transitory, tangible “storage” media, terms such as computer or machine “readable medium” refer to any medium that participates in providing instructions to a processor for execution.

Hence, a machine readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The computer system 1701 can include or be in communication with an electronic display 1735 that comprises a user interface (UI) 1740 for providing, for example, genetic data, including for example, base sequence strings, or reads in various syntaxes, sequence alignments. Examples of UIs include, without limitation, a graphical user interface (GUI) and web-based user interface.

Methods and systems of the present disclosure can be implemented by way of one or more algorithms. An algorithm can be implemented by way of software upon execution by the central processing unit 1705. The algorithm can, for example, encrypt data, translate genetic reads, analyze, interpret, align, and assemble various data including but not limited to sequence data, working data, meta data, sample data, contig data.

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

1.-55. (canceled)

56. A method for storing sequence base data in a multi-level cell (MLC) memory device, the MLC memory device comprising memory cells, each of the memory cells configured to store at least two bits, the method comprising, in a memory cell:
(a) setting two of the at least two bits to 00 to represent a base of a first type;
(b) setting two of the at least two bits to 01 to represent a base of a second type;
(c) setting two of the at least two bits to 10 to represent a base of a third type; or
(d) setting two of the at least two bits to 11 to represent a base of a fourth type.

57. The method of claim 56, wherein the sequence base data represents one or more polynucleotides, each of the polynucleotides comprising one or more bases, each of the one or more bases being one of at least four possible bases.

58. The method of claim 57, wherein the one or more polynucleotides are DNA or RNA.

59. The method of claim 56, wherein said at least two bits comprise at least three bits.

60. The method of claim 56, further comprising:
(1) setting three of the at least three bits to 000 to represent the base of the first type;
(2) setting three of the at least three bits to 001 to represent the base of the second type;
(3) setting three of the at least three bits to 010 to represent the base of the third type;
(4) setting three of the at least three bits to 011 to represent the base of the fourth type;
(5) setting three of the at least three bits to 100 to represent a base of a fifth type;
(6) setting three of the at least three bits to 101 to represent a base of a sixth type;
(7) setting three of the at least three bits to 110 to represent a base of a seventh type; and
(8) setting three of the at least three bits to 111 to represent a base of an eighth type.

61. The method of claim 60, wherein the sequence base data represents one or more polynucleotides, each of the polynucleotides comprising one or more bases, each of the one or more bases being one of four different native bases, a methylated base, an oxidated base, or an abasic location.

62. The method of claim 61, wherein the one or more polynucleotides are DNA or RNA.

63. The method of claim 56, wherein the MLC memory device comprises a flash memory, a phase-change memory, or a resistive memory.

64. A method for encrypting biological sequence data, the method comprising:
(a) identifying a normal level of variance in the biological sequence data; and
(b) introducing a second level of variation into the biological sequence data, the second level of variation comparable to the normal level of variance, such that the biological sequence data is indistinguishable with respect to the normal level of variance.

65. The method of claim 64, further comprising communicating the second level of variance using an encryption method.

66. The method of claim 64, further comprising
(a) encrypting information related to the subject using a first encryption scheme; and
(b) encrypting the biological sequence data using a second encryption scheme, wherein the second encryption scheme is different from the first encryption scheme.

67. The method of claim 66, wherein the second encryption scheme comprises a less extensive encryption than the first encryption scheme.

68. The method of claim 67, wherein the second encryption scheme comprises chaffing and winnowing.

69. The method of claim 67, wherein the first encryption scheme and the second encryption scheme use a public key infrastructure.

70. The method of claim 67, wherein the first encryption scheme uses a first public key infrastructure and the second encryption scheme uses a second public key infrastructure different from the first public key infrastructure.

71. A method for storing sequence base data comprising at least four possible bases, the method comprising:
(a) providing a three-dimensional table structure in computer memory, which three-dimensional table structure is configured to store the sequence base data, wherein (i) a first dimension of the three-dimensional table structure stores information representing most probable measured bases of the genetic sequence base data; (ii) a second dimension of the three-dimensional table structure stores information representing potential bases of the genetic sequence base data; and (iii) a third dimension of the three-dimensional table structure stores information representing a base count probability for each of the at least four possible bases of the sequence base data;
(b) storing probabilities corresponding to an intersection of the first dimension, the second dimension, and the third dimension in the three-dimensional table structure.

72. The method of claim 71, further comprising providing a second three-dimensional table structure in computer memory, the second three-dimensional table structure configured to store information representing the potential bases; and storing in the second three-dimensional table structure the most probable measured bases of the sequence base data and a second most probable measured bases of the sequence base data.

73. The method of claim 72, further comprising providing a third three-dimensional table structure in computer memory, the third three-dimensional table structure configured to store information representing the potential bases; and storing in the third three-dimensional table structure the most probable measured bases of the sequence base data, the second most probable measured bases of the sequence base data, and a third most probable measured bases of the sequence base data.

74. The method of claim 71, wherein the potential bases represent one or more polynucleotides, each of the polynucleotides comprising a set of each of four possible bases and at least one of a methylated base, an oxidated base, and an abasic site.

75. The method of claim 74, wherein the one or more polynucleotides are DNA or RNA.
